(19) United States
(12) Patent Application Publication
(10) Pub. No.: US 2003/0012772 A1
(43) Pub. Date: Jan. 16, 2003

(54) Methods for improving cell line activity in immunoisolation devices

(75) Inventors: Richard Schneiderman, Sandy Hook, CT (US); Revati J. Tatake, Sandy Hook, CT (US); Randall Wilber Barton, Farmington, CT (US)

Correspondence Address: BOEHRINGER INGELHEIM CORPORATION, 900 RIDGEBURY ROAD, P O BOX 368, RIDGEFIELD, CT, 06877, US

(73) Assignee: Boehringer Ingelheim Pharmaceuticals, Inc., Ridgefield, CT

(21) Appl. No.: 10/166,146

(22) Filed: Jun. 10, 2002

Related U.S. Application Data
(60) Provisional application No. 60/296,936, filed on Jun. 8, 2001.
(60) Provisional application No. 60/296,935, filed on Jun. 8, 2001.

(51) Int. Cl. A61K 45/00; C12N 5/06

(52) U.S. Cl. 424/93.7; 435/325

(57) ABSTRACT

Methods for maintaining and improving the secretory activity of cells housed in immunoisolation devices.

### RELATED APPLICATIONS

The present application claims priority benefit of U.S. Provisional Application Nos. 60/296,936 and 60/296,935, both filed on Jun. 8, 2001.

### BACKGROUND OF THE INVENTION

1. Field of the Invention

The present invention relates to methods useful for maintaining and improving the biological activities, in particular secretory activity, of cells housed within an immunoisolation device.

2. Background of the Related Art

Conventional treatment of functional deficiencies of biological organs has centered on the replacement of identified normal secreted products of the deficient organ with natural or synthetic pharmaceutical compositions. Many clinical conditions and disease states can be ameliorated or remedied by supplying to the patient one or more biologically active agents produced by living cells. Examples of disease or deficiency states whose etiologies include loss of secretory organ or tissue function include, without limitation: (a) diabetes, wherein the production of insulin by the islets of Langerhans in the pancreas is impaired or lost; (b) paralysis agitans (more commonly known as “Parkinson's disease”), which is characterized by a lack of the neurotransmitter dopamine within the striatum of the brain; (c) amyotrophic lateral sclerosis, a disease involving the degeneration of motor neurons of the spinal cord, brain stem, and cerebral cortex; (d) hypoparathyroidism which involves the loss of the production of parathyroid hormone, which causes calcium levels to drop, resulting in muscular tetany; (e) anemia, which is characterized by the loss of production of red blood cells secondary to a deficiency in the production of erythropoietin.

Clinical therapy also often entails the administration of biologically active moieties even without an underlying deficiency in tissue production of the moiety. For example, lymphokines and cytokines are frequently administered to patients to enhance their immune system or to act as anti-inflammatory agents. Likewise, trophic factors, such as nerve growth factor and insulin-like growth factors 1 and 2, have also been advocated for clinical use. Trophic and growth factors may be used to prevent neurodegenerative conditions, such as Huntington's and Alzheimer's diseases, and adrenal chromaffin cells, which secrete catecholamines and enkephalins, may be used to treat pain.

In many disease and deficiency states, an affected tissue or organ is one which normally functions in a manner responsive to fluctuations in the levels of specific metabolites, products, and electrolytes, thereby maintaining homeostasis. For example, the parathyroid gland normally modulates production of parathyroid hormone in response to fluctuations in serum calcium, and beta cells in the pancreatic islets of Langerhans normally modulate the production of insulin in response to fluctuations in serum glucose. It is therefore understandable that conventional modes of administration of exogenous biologically active agents, as by, for example injection, are often not optimal, given the numerous fluctuations in need for the biological agent that may occur during a day. This is true with respect to numerous disease states, including, but not limited to, diabetes and anemia.

Diabetes mellitus is a chronic disorder of fat, carbohydrate, and protein metabolism. It is characterized by an under-utilization of glucose, and an absolute or relative insulin deficiency. Diabetes is treated by correcting insulin concentrations in the body in such a manner that the patient has as normal or as nearly normal carbohydrate, fat and protein metabolism as possible. Optimal therapy has been found to be effective at preventing most acute effects of diabetes, and to greatly delay the chronic effects as well. Treatment for diabetes is still centered around self-injection of exogenous insulin once or twice daily, or in the case of non-severe diabetes wherein the islets still maintain the potential to secrete insulin, the use of drugs that stimulate insulin secretion such as the sulfonylureas. Exogenous insulin may be isolated by non-recombinant methods as from the purification of insulin from freshly isolated porcine or bovine pancreas, or by employment of recombination techniques. Daily injections of insulin, the accepted treatment for diabetes mellitus, cannot compensate for the rapid, transient fluctuations in serum glucose levels produced by strenuous exercise. Failure to provide adequate compensation may lead to complications of the disease state.

Anemia is associated with numerous biological perturbations, including chronic renal failure, cancer, and human immunodeficiency virus infections. It is known that injections of erythropoietin (EPO) are particularly useful for increasing red blood cell count. EPO-secreting cells are destroyed in a number of these disease states, in particular chronic renal failure. While repeated injections of EPO have been found to be useful, strict adherence to dosage schedules has been found to be difficult in many patients. Patients using EPO not infrequently demonstrate less than optimal blood hematocrits.

It is recognized by those of ordinary skill in the art that many disease states could be treated in a more physiologic fashion if tissue from other animals, human and/or non-human, could be transplanted into the person suffering from the disease. A major problem with allogeneic transplants is that the availability of such transplants is limited, and the host into which the transplant is made must typically be kept immunosuppressed for a lifetime to prevent destruction of the transplant by the host's immune system. While xenogeneic transplants greatly improve the availability of tissue for transplantation, xenogeneic material, there have been no successful long-term engraftments to date irrespective of the degree of immunosuppression. It is both undesirable and expensive to maintain a patient in an immunosuppressed state for a substantial period of time. Syngeneic transplants also suffer from drawbacks. For one, the person suffering from the disease state often does not have the cells available to donate. Secondly, the disease state may result from an autoimmunity that is destructive to the cells that will be transplanted. Further, culturing of cells outside of the body typically requires mutating the cells to provide for unregulated growth, leading to the problems associated with the transplantation of malignant material.

An alternate approach to tissue transplantation that has been suggested involves using a bioartificial implant known as an immunoisolation device. An immunoisolation device is a device or material which houses cells or tissue and allows diffusion of nutrients, waste materials, and secreted products, but blocks the cellular effectors of immunological rejection. An immunoisolation device may, or may not, block molecular effectors. Generally in immunoisolation devices a selectively permeable membrane acts to protect the transplanted cells, tissue or organ from being destroyed by the host's immune system. For example, the in vivo treatment of diabetes with peritoneal implants of encapsulated islets has been reported by several research groups (See, e.g., U.S. Pat. No. 5,262,055 to Bae et al. (1993); U.S. Pat. No. 5,427, 940 to Newgard (1992); Lum et al., Diabetes 40: 1511 (1991); Maki et al., Transplantation 51: 43 (1991); Robertson, Diabetes 40: 1085 (1991); Colton et al., J. Biomech. Eng. 113: 152 (1991); Scharp et al., Diabetes 39: 515 (1990); Reach, Intern. J. Art. Organs 13: 329 (1990). Immunoisolation devices are even employed with syngeneic or autologous materials to prevent migration of the cells out of the device, particularly if the cells have been altered in vitro to become immortalized.

Many biocompatible materials, such as lipids, polycations and polysaccharides, have been used to encapsulate living cells and tissues and to isolate the same from the immune system. Cells have particularly been encapsulated with alginates (See, e.g., U.S. Pat. No. 5,976,780 to Shah (Issued: Nov. 2, 1999) and U.S. Pat. No. 6,023,009 to Stegemann et al. (Issued: Feb. 8, 2000)). Likewise, many other structures have been employed including extravascular diffusion chambers, intravascular diffusion chambers, and intravascular ultrafiltration chambers (See, Scharp, D. W., et al., World J. Surg. 8: 221 (1984)).

U.S. Pat. No. 5,869,077 to Dionne et al. (Issue Date: Feb. 9, 1999) describes a biocompatible immunoisolatory vehicle suitable for long-term implantation into individuals comprising a core which contains a biological moiety, such as a cell, either suspended in a liquid medium or immobilized within a hydrogel or extracellular matrix, and a surrounding or peripheral region of perselective matrix or membrane which does not contain the isolated biological moiety and which protects the biological moiety from immunological attack, but has a molecular weight cutoff (advantageously 50 kD to 2000 kD) to permit passage of molecules between the patient and the core. The jacket of such device may be fabricated from materials such as polyvinylchloride, polyacrylonitrile, polymethylmethacrylate, polyvinyldifluoride, polyolefins, polysulfones and celluloses. Likewise, PCT/US99/08628 to Powers et al. teaches immunoisolation devices comprising alginate coatings, and cells seeded into semipermeable fibers.

A commercially available implantable immunoisolation device is the TheraCyte® device (TheraCyte Inc., Irvine, Calif.). The device is designed for subcutaneous or intraperitoneal implantation and is said to enable allogeneic cell transplants without immunosuppression, and to protect xenogeneic transplants with conventional immunosuppression. The device comprises an outer vascularizing membrane of polytetrafluoroethylene (PTFE) 15 μm thick and having 5 μm pore size, and an inner, cell impermable PTFE membrane 30 μm thick and having 0.4 μm pore size. The outer membrane is said to be vascularizing, thus preventing the common problem of fibrotic encapsulation usually encountered with bioimplantable devices.

Another commercially available implantable immunoisolation device is manufactured by VivoRx® and comprises microcapsules with purified alginate containing a high glucuronic acid content. The microbeads are said to prevent the formation of fibroblasts for a significant period of time.

It is unfortunate that immunoisolation devices have frequently been found to be less than effective due to overgrowth or rapid senescence of cells in the device.

When an implant is placed in a host, the typical biological response by the recipient is the formation of a fibrotic capsule, comprising flattened macrophages, foreign body giant cells and fibroblasts, around the device. The fibrotic capsule may deprive the encapsulated cells of the life-sustaining exchange of nutrients and waste products with tissues of a recipient. According to Brauker et al., U.S. Pat. No. 5,314,471 (Issued: May 24, 1994) the problem due to the fibrotic capsule may be overcome by improving the metabolic transit value of the device, as well as by including an angiogenic material in the device that stimulates the growth of vascular structures by the host.

Even if the cells are permitted to grow and survive initial transplant, and the subsequent formation of the fibrotic capsule, because the cells employed in the devices frequently are undergoing rapid cell division, the increasing oxygen and nutrient demand within the encapsulation, as well as an increase in metabolic wastes, adversely impact the survivability of the cells. That is, immunoisolation devices often fail because their dimensions are such that the enclosed cells cannot receive enough nutrients, especially oxygen. When the cells are starved of oxygen, their metabolism declines and they lose the ability to secrete the polypeptide or other material that is desired.

Many cells included in immunoisolation devices are cells that have been immortalized in vitro in order to culture the same, both for the purpose of increasing cell number, as well as to allow recombinant techniques to be employed to transform the cells in a manner such that they will express materials useful for the treatment of the disease state. Beyond the problem of cell growth in the immunoisolation device, a common problem associated with such cells lines is their phenotypic instability. For example, cells responsive to physiological concentrations of secretagogues in vitro frequently become responsive to subphysiological concentrations of the secretagogue over time when placed into an immunoisolation device.

It is not easy to discern all of the reasons why some cell lines adapt better in one immunoisolation device versus another. It is known, however, that certain cell lines are far more robust in surviving within immunoisolation devices, and far more efficient at performing their secretory duties in such devices, than others. Heretofore, isolation and identification of these cell lines has been happenstance.

There is a great need, therefore, for providing immunoisolation systems comprising cell lines and immunoisolation devices, that are maximized for cell survival and the desired activity of the cell line, such as a particular polypeptide secretion. Such systems could significantly improve the treatment of numerous disease states, allowing for the avoidance of onerous dosage schedules and permitting tailored administration of the treatment modality with respect to physiological need.

### SUMMARY OF THE INVENTION

The present invention is related to in vitro and in vivo selection methods to derive cell lines that are better adapted to survive in an immunoisolation device while retaining optimal function, or to derive cell lines that have enhanced biological properties as compared with the parental cell line.

Prior to the present invention, immunoisolation devices were loaded with cells and cell lines identified to have the particular functional activity desired, without regard to the robustness of the cells with respect to the environment of the immunoisolation device. Cells and cell lines isolated for having a particular functionality were put into immunoisolation devices and evaluated for growth and/or secreted protein production. No method or procedure for selecting cells with optimal functional characteristics in the immunoisolation device environment was undertaken.

There are a host of unknown factors that affect the growth and activity of cells in immunoisolation devices. These large number of factors argue against in vitro models to determine cells most likely to survive in the immunoisolation device, and may be one of the reasons that the prior art has not attempted in the past to maximize cell “robustness” in the immunoisolation device environment. Many factors, including response to hypoxia and cell density, as well as response to inflammatory mediators to which the cells may be exposed, affect the activity and growth of cells housed within an immunoisolation device. For example, it is known that immediately following sub-cutaneous implantation, cells in an immunoisolation device experience a prolonged period of hypoxia until the device becomes vascularized. Cells that can function under these hypoxic conditions would be desirable.

The present inventors have designed a method of selecting for cells that can thrive in the environment of an immunoisolation/encapsulation device when such device is implanted, for example, subcutaneously. This is accomplished by implanting the device containing the cells, explanting the device, and then recovering the cells and expanding them in vitro. Alternatively, the device containing the cells is cultured and then the cells are recovered and expanded in vitro. In both methods, the cells then are re-cultured or re-implanted in another immunoisolation device, advantageously of similar construct, and monitored for function. As would be understood by one of ordinary skill in the art from this description, this procedure can be repeated numerous times if deemed desirable or necessary.

Also disclosed is a system for the delivery of therapeutic proteins comprising cells that either naturally or by genetic engineering (as by transfection of vector containing exogenous DNA, of homologous or heterologous origin, encoding for a desired protein) secrete the protein or product of interest that are selected by the above method and a implantable encapsulation device. Transformation of cells to be used in such a system may be effectuated by any of the methods well known to those of ordinary skill in the art, as described, for example, in Sambrook et al., Molecular Cloning: A Laboratory Manual, 2d Ed., Cold Spring Harbor Press, Cold Spring Harbor, N.Y. (1989). As would be understood by the skilled artisan, such methods include, without limitation, calcium phosphate transfection, DEAE-dextran mediated transfection, microinjection, cationic lipid-mediated transfection, electroporation, transduction, scrape loading, ballistic introduction or infection.

### BRIEF DESCRIPTION OF THE DRAWINGS

FIG. 1 is a diagrammatic representation of an in vivo method of the present invention for improving the survivability of cell lines in an immunoisolation device.

FIG. 2 is a diagrammatic representation of an in vitro method of the present invention for improving the survivability of cell lines in an immunoisolation device.

FIG. 3 is a graph of the percent hematocrit, over time, in rats subcutaneously implanted with a Theracyte® immunoisolation device comprising rat vascular smooth muscle cells transformed with an appropriate vector to eventuate in the secretion of erythropoeitin, the cells included in the device being isolated either from primary culture, or by the method of the present invention.

### DETAILED DESCRIPTION OF THE INVENTION

1. Definitions:

The following definitions are provided to facilitate understanding of certain terms used herein:

By “allogeneic” it is meant that two cells or cell lines or a cell line and an organism are derived from individuals of the same species that are sufficiently unlike genetically to interact antigenically.

By “autograft” it is meant a graft taken from one part of the body and placed in another site of the body of the same individual.

By “autologous” it is meant cells, tissues, organs, DNA, etc., derived from the same individual.

By “cells” it is meant to include cells in any form, including, but not limited to, cells retained in tissue, cell clusters and individually isolated cells.

By “cell line” it is meant cells capable of stable growth in vitro for many generations.

By “clone” it is meant a population of cells derived from a single cell or common ancestor by mitosis.

By “con-specific” it is meant that two cells or cell lines or a cell line and an organism are from the same animal species.

By “exogenous” material it is meant material that has been introduced into a cell, organism etc. that originated outside of the same.

By “heterologous” it is meant derived from tissues or DNA of a different species.

By “homologous” it is meant derived from tissues of DNA of a member of the same species.

By “immunoisolation device” it is meant a device or material which houses cells or tissue and allows diffusion of nutrients, waste materials, and secreted products, but blocks the cellular effectors of immunological rejection. An immunoisolation device may, or may not, block molecular effectors. Generally in immunoisolation devices a selectively permeable membrane acts to protect the transplanted cells, tissue or organ from being destroyed by the host's immune system.

By “isolate” material, it is meant changing the environment of the material or removing a material from its original environment, or both. For example, when a polynucleotide or polypeptide is separated from the coexisting materials of its natural state, it is “isolated.”

By “recombinant” or “engineered” cell it is meant a cell into which a recombinant gene has been introduced through the hand of man. Recombinantly introduced genes may be in the form of a cDNA gene (i.e., lacking introns), a copy of a genomic gene (i.e., including introns with the exons), genes produced by synthetic means, and/or may include genes positioned adjacent to a promoter, or operably linked thereto, not naturally associated with the particular introduced gene.

By “replicon” it is meant any genetic element (e.g., plasmid, chromosome, virus) that functions as an autonomous unit of DNA replication in vivo, i.e. capable of replication under its own control.

By “secretagogue” it is meant a substance that induces secretion from cells.

By “syngeneic” it is meant that two cells or cell lines or a cell line and an organism are from the same individual.

By “transformed cell” it is meant a cell into which exogenous or heterologous DNA has been introduced. The transforming DNA may or may not be integrated (covalently linked) into chromosomal DNA making up the genome of the cell. The transforming DNA may be maintained on an episomal element such as a plasmid.

By “transfection” it is meant the introduction of a nucleic acid sequence into a target cell.

By “variant” it is meant a sequence, such as a polynucleotide or polypeptide, that differs from another sequence, but retains essential properties thereof, that is, properties for which the sequence is utilized in its application (e.g., promoting expression, cleaving a bond, etc.). For example, a variant of a polynucleotide may differ in nucleotide sequence by one or more substitutions, additions, and deletions, from the reference polynucleotide. By “variant” it is also meant to include fragments of a full length sequence that retains essential properties thereof.

By “vector” it is meant a replicon, such as a plasmid, phage or cosmid, used for the transformation of cells in gene manipulation. Vectors may include nucleotide molecules from different sources which have been artificially cut and joined.

By “xenogeneic” is meant that two cells or cell lines or a cell line and an organism are from individuals of a different species.

2. The Method

The present invention overcomes many of the problems associated with the prior art's use of immunoisolation devices for the amelioration of disease states associated with a deficiency in production of a cell secretory product.

While the prior art has concentrated on deficiencies in the construct of immunoisolation devices to explain the general poor performance of immunoisolation systems in the amelioration of disease states associated with a deficiency in cell secretory product, the present inventors have recognized that a far more significant cause for the lack of performance of these systems relates to the failure of investigators to isolate cells and cell lines that are maximized for survival in the immunoisolation device under the conditions in which it is to be employed (e.g., subcutaneously). Recognizing that a large number of factors impact on both the survivability and optimization of cell functions in such devices, the present inventors have designed methods for selecting cells that can survive and function optimally in immunoisolation devices.

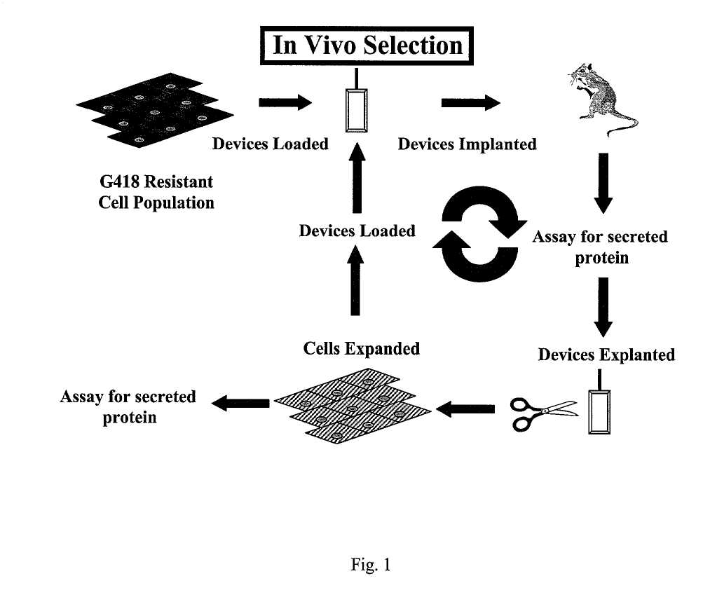

Turning to FIG. 1, there is shown a diagrammatic representation of an in vivo method of the present invention for improving the survivability of cell lines and the efficiency of their functionality in an immunoisolation device.

As shown, the specific cell population, whether native or genetically engineered, is loaded (A) into the device which is then implanted subcutaneously (B) into an animal selection host believed to react to the immunoisolation device in a manner similar to the recipient in which cells are ultimately to be placed in an immunoisolation device. As would be understood by one of ordinary skill in the art, other implantation modes could also be employed if the device was ultimately employed in another location in the body of the intended recipient (e.g., the device could be implanted intraperitoneally). The functional integrity of the cells in the device may be monitored post-implantation over time. Typically, the primary functional readout is the blood level of a desired secreted protein as a function of time (C). After a time post-implantation, typically in the range of days or weeks, the device is explanted (D) and the cells within the device are recovered. The cells are then expanded in vitro (E) and assayed for the desired secreted protein to assure retention of functionality (F). These cells then are loaded into new devices (G) and implanted either once more into animal selection host or into the intended recipient in need of the product produced by the cells. As would be understood by one of ordinary skill in the art, this method can be re-iterated numerous times to maximize the cell line to be implanted prior to its incorporation into the ultimate immunoisolation device that is to be employed to treat the disease state of the recipient.

The method of FIG. 1 results in the isolation of cells that typically secrete the protein of interest in detectable levels significantly earlier in an animal with a secondary implant than is seen in animals having the primary implant. The magnitude of the level of secretion may also be greater in the animal with secondary implant as compared to the animal with the primary implant.

In one embodiment of the present invention there is disclosed an in vivo method for optimizing cell survival in an immunoisolation device implantation in a recipient animal, said method comprising the steps of: (a) loading cells into a first immunoisolation device; (b) implanting the immunoisolation device into a host animal; (c) removing the immunoisolation device from said host animal after a period of time; (d) unloading the cells from the removed immunoisolation device; (e) expanding the unloaded cells on medium supporting growth of said cells; (f) loading the expanded cells into another immunoisolation device; and (g) optionally repeating steps (b)-(f) for one or more times. The loaded immunoisolation device of step (f) contains cell lines optimized for survival in an immunoisolation device implantation in the recipient animal. The implantation of the immunoisolation device into said host at step (b) preferably is performed in a manner consistent with the intended method of implantation with respect to said recipient. The host animal and the recipient animal may be of the same or different species. Cells used in the method may be allogeneic, xenogenic, con-specific, or syngeneic to said recipient. The cells may be naturally occurring cells or may be cells of recombinant origin, such as those transformed by transfection by a vector comprising heterologous and/or homologous polynucleotide(s). The cells may be isolated from a common clone. In most applications, the cells will secrete a polypeptide, or variant thereof, needed for the homeostasis of said recipient, and in many applications secretion will be inducible by way of a secretagogue. The period of time in step (c) is advantageously in the range of days, weeks or months. Advantageously steps are repeated at least twice.

In another embodiment of the present invention there is provided an in vivo method for selecting cells with optimal desired functionality in an immunoisolation device implantation in a recipient animal, said method comprising the steps of: (a) loading cells having the desired functionality into a first set of immunoisolation devices; (b) implanting the first set of immunoisolation devices into a plurality of host animals; (c) monitoring said host animals for the cellular functionality; (d) removing the immunoisolation devices from the host animals suggesting a predetermined level of cellular functionality; (e) unloading the cells from the removed immunoisolation device onto a plurality of medium supports supporting growth of the unloaded cells; (f) expanding the unloaded cells on the medium supports; (g) determining the medium supports that contain cells having a predetermined level of desired cellular functionality; (h) loading the expanded cells having said predetermined level of desired cellular functionality into another immunoisolation device; and (i) optionally repeating steps (b)-(h) for one or more times. The loaded immunoisolation device of step (h) contains cell lines having optimal desired functionality for immunoisolation device implantation into said recipient animal. Preferably the implantation of said immunoisolation device into the host animals at step (b) is performed in a manner consistent with the intended method of implantation with respect to the recipient. The host animals and said recipient animal may be of the same or different species. Typically, each of the first set of immunoisolation devices of step (a) are implanted into a separate host animal in step (b). Typically, monitoring of the host animals at step (c) entails monitoring of blood levels of a product. Advantageously, the cells from each removed immunoisolation device in step (e) are unloaded onto a separate medium support. The cells may be allogeneic, xenogeneic, con-specific or syngeneic to the recipient. The cells may be naturally occurring cells or may be cells of recombinant origin, such as those transformed by transfection by a vector comprising heterologous and/or homologous polynucleotide(s). The cells may be isolated from a common clone. In most applications, the cells will secrete a polypeptide, or variant thereof, needed for the homeostasis of said recipient, and in many applications secretion will be inducible by way of a secretagogue. The period of time in step (c) is advantageously in the range of days, weeks or months. Advantageously steps (b)-(h) are repeated at least twice.

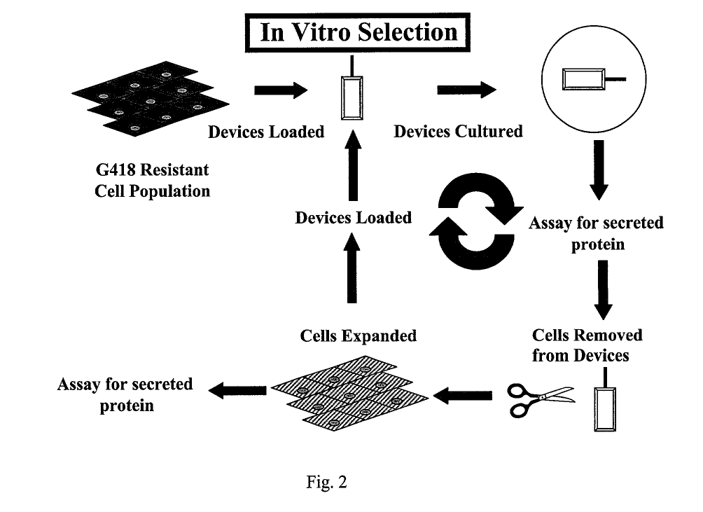

Turning to FIG. 2, there is shown a diagrammatic representation of an in vitro method of the present invention for improving the survivability of cell lines and the efficiency of their functionality in an immunoisolation device.

As shown, the specific cell population, whether native or genetically engineered, is loaded (A) into the device which is then cultured (B). The functional integrity of the cells in the device may be monitored post-culture over time. Typically, the primary functional readout is the level of a desired secreted protein as a function of time (C). After a time post-culture, the cells within the device are recovered (D). The cells may be cultured in the devices as long as the product is produced by the cells, i.e., days, weeks months, , years. Production of such product may be monitored by methods known in the art, including but not limited to radioimmunoassay. The cells are then expanded in vitro (E) and assayed for the desired secreted protein to assure retention of functionality (F). These cells then are loaded into new devices (G) and cultured again or implanted into the intended recipient in need of the product produced by the cells. As would be understood by one of ordinary skill in the art, this method can be re-iterated numerous times to maximize the cell line to be implanted prior to its incorporation into the ultimate immunoisolation device that is to be employed to treat the disease state of the recipient.

The method of FIG. 2 results in the isolation of cells that typically secrete the protein of interest in detectable levels significantly earlier in an animal with a cultured implant than is seen in animals having the primary implant. The magnitude of the level of secretion may also be greater in the animal with a cultured implant as compared to the animal with the primary implant. In addition, there may be other biological properties that can be selected for that will improve the performance of the selected cells.

In one embodiment of the present invention there is disclosed an in vitro method for optimizing cell survival in an immunoisolation device implantation in a recipient animal, said method comprising the steps of: (a) loading cells into a first immunoisolation device; (b) culturing the immunoisolation device in a culture vessel; (c) removing the immunoisolation device from the culture vessel after a period of time; (d) unloading the cells from the removed immunoisolation device; (e) expanding the unloaded cells on medium supporting growth of said cells; (f) loading the expanded cells into another immunoisolation device; and (g) optionally repeating steps (b)-(f) for one or more times. The loaded immunoisolation device of step (f) contains cell lines optimized for survival in a cultured immunoisolation device. The culture of the immunoisolation device at step (b) preferably is performed in a manner consistent with the culture conditions of the cells. Cells used in the method may be allogeneic, xenogenic, con-specific, or syngeneic to said recipient. The cells may be naturally occurring cells or may be cells of recombinant origin, such as those transformed by transfection by a vector comprising heterologous and/or homologous polynucleotide(s). The cells may be isolated from a common clone. In most applications, the cells will secrete a polypeptide, or variant thereof, needed for the homeostasis of said recipient, and in many applications secretion will be inducible by way of a secretagogue. The cells may be cultured in the devices as long as the product is produced by the cells. Production of such product may be monitored by methods known in the art, including but not limited to radioimmunoassay. Advantageously steps are repeated at least twice.

In another embodiment of the present invention there is provided an in vitro method for selecting cells with optimal desired functionality in an immunoisolation device implantation in a recipient animal, said method comprising the steps of: (a) loading cells having the desired functionality into a first set of immunoisolation devices; (b) culturing the first set of immunoisolation devices; (c) monitoring said cultured devices for the cellular functionality; (d) removing the immunoisolation devices from culture suggesting a predetermined level of cellular functionality; (e) unloading the cells from the removed immunoisolation device onto a plurality of medium supports supporting growth of the unloaded cells; (f) expanding the unloaded cells on the medium supports; (g) determining the medium supports that contain cells having a predetermined level of desired cellular functionality; (h) loading the expanded cells having said predetermined level of desired cellular functionality into another immunoisolation device; and (i) optionally repeating steps (b)-(h) for one or more times. The loaded immunoisolation device of step (h) contains cell lines having optimal desired functionality for immunoisolation device implantation into said recipient animal. Preferably the culture of said immunoisolation device at step (b) is performed in a manner consistent with the culture conditions of the cells. Typically, monitoring of the cells in the cultured devices at step (c) entails monitoring of levels of a product. Advantageously, the cells from each removed immunoisolation device in step (e) are unloaded onto a separate medium support. The cells may be allogeneic, xenogeneic, con-specific or syngeneic to the recipient. The cells may be naturally occurring cells or may be cells of recombinant origin, such as those transformed by transfection by a vector comprising heterologous and/or homologous polynucleotide(s). The cells may be isolated from a common clone. In most applications, the cells will secrete a polypeptide, or variant thereof, needed for the homeostasis of said recipient, and in many applications secretion will be inducible by way of a secretagogue. The cells may be cultured in the devices as long as the product is produced by the cells. Production of such product may be monitored by methods known in the art, including but not limited to radioimmunoassay. Advantageously steps (b)-(h) are repeated at least twice.

And in yet another embodiment of the present invention, there is provided an immunoisolation system comprising: (a) cells selected by the disclosed methods and (b) an immunoisolation device, wherein the selected cells are housed within an immuno-isolation device.

### 3. EXAMPLES

The following examples are provided to illustrate the invention, but not to limit its scope. Other variants of the invention will be readily apparent to one of ordinary skill in the art, and are encompassed by the appended claims.

### Example 1

Rat smooth muscle cells expressing erythropoietin were produced as described in Lejnieks et al., Blood 92(3): 888-893 (1998). In brief, retroviral vector LrEpSN was made by inserting an EcoRI-BamHI fragment of the rat Epo cDNA into LXSN. A PA317 retroviral packing cell line was used.

Rat smooth muscle cell cultures were prepared by enzymatic digestion of a male Fisher 344 rat aorta. Cells were characterized by positive staining for muscle cell-specific actins with HHF35 antibody and staining negative for von Willebrand factor. Primary cultures of rat smooth muscle cells and PA317-LrEpSN were grown in DulbeccoNogt modified Eagle's medium (“DMEM”) supplemented with 10% fetal bovine serum in humidified 5% CO₂ at 37° C. Early passage smooth muscle cells were exposed to 16-hour virus harvests from PA317-LrEpSN for a period of 24 hours in the presence of polybrene. Vascular smooth muscle cells infected with LrEpSN were selected in 1 mg/ml G-418 antibiotic. Selected cells were seen to secrete about 6.7 mU/24 h per 10⁵ cells of erythropoietin.

Rat vascular smooth muscle cells expressing erythropoietin were loaded into a Theracyte® immunoisolation device. The device with loaded cells was then implanted subcutaneously into a rat on its dorsal side to form a primary implant. Hematocrits were measured to monitor the secretion of erythropoietin from the primary implant every 10 days up to 70 days. After day 70 the primary implant was explanted and the cells within the immunoisolation device were recovered. The cells were then expanded in vitro until a sufficient number of cells was obtained for re-implantation. The resulting cells were then loaded into two new Theracyte® immunoisolation devices, and one of each device was implanted on the dorsal side of two new rats. The secretion of erythropoietin was once again monitored by measurement of hematocrits every 5-10 days for 35 days.

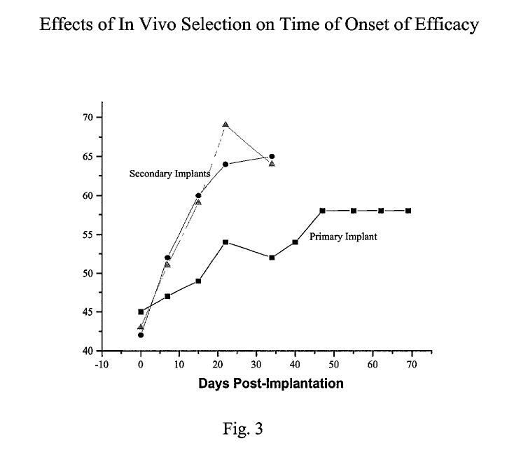

As seen in FIG. 3, the onset of an increased hematocrit occurred substantially earlier in rats receiving the secondary implants as opposed to the rat that received the primary implant. Moreover, the magnitude of increased hematocrit was significantly greater for rats with secondary implants as opposed to the rat with the primary implant for each time point measured. By day thirty-five, hematocrit levels were approximately twenty percent lower in the rat receiving the primary implant than in the rats receiving the secondary implants.

### Example 2

Alternatively to implanting devices loaded with cells into an animal, devices were loaded with glucose-responsive insulin-producing transformed cells as described in Example 3 (for example, Rat 22, U-20S, A-498 or SHP-77) and cultured for 12 to 15 months. The secretion of insulin was monitored approximately every 2 weeks by insulin radioimmunoassay. After 12 to 15 months, the cells were recovered from the devices and expanded in vitro. The recovered cells were found to produce insulin in a glucose-responsive manner as determined by radioimmunoassay.

### Example 3

Barry et al., Human Gene Therapy 12: 131 (Jan. 20, 2001), describe retroviral vectors encoding glucose-responsive promoters driving furin expression used to provide an amplified, glucose-regulated secretion of insulin. The LhI*TFSN virus construct encodes a glucose-regulatable rat transforming growth factorα (TGFα) promoter controlling murine furin expression with a viral long terminal repeat promoter (LTR) driving constitutive expression of furin-cleavable human proinsulin. When such constructs are transduced into vascular smooth muscles cells, the cells are seen to respond to physiological glucose concentrations. The furin-cleavable human proinsulin was obtained by mutating human proinsulin cDNA to encode furin-cleavable sites (Hosaka et al., J. Biol. Chem. 255: 12127 (1991); Groskruetz et al., J. Biol. Chem. 269: 6241 (1994); Gros et al., Gene Ther. 8: 2249 (1997)). The selectable neo gene (bacterial neomycin phosophotransferase) marker in such construct is expressed from and driven by the simian virus 40 promoter (SV40).

Lhl*TFSN was used to transform a number of cell lines. The cells were placed into a Theracyte® immunoisolation device. Several cell lines were identified that demonstrated high production of insulin upon secondary implant using the above-described methodology. The identified numerous human cell lines are well adapted to in vitro culture and genetic modification, and were found to be adapted for growth in immunoisolation devices. These cells comprise:

TABLE I

| CELL | | CELL | TISSUE |
|---|---|---|---|
| DESIGNATION | TISSUE TYPE | DESIGNATION | TYPE |
| HEPM | Palatal Mesenchyme | CRL-1486 | Fibroblast |
| U-2OS | Bone | FITB-96 | Epithelia |
| A-498 | Kidney | HTB-44 | Epithelia |
| NCI-H441 | Lung | HTB-174 | Epithelia |
| SHP-77 | Lung | CRL-2195 | Epithelia |

### 4. Scope of the Invention

While the invention has been described with respect to preferred embodiments, those skilled in the art will readily appreciate that various changes and/or modifications can be made to the invention without departing from the spirit or scope of the invention as defined by the appended claims. The present disclosure is to be considered as in all respects illustrative and not restrictive, the scope of the invention further being indicated by the appended Claims, and all changes which come within the meaning and range of equivalency are intended to be embraced therein.

All documents cited herein are incorporated by reference in their entirety.

What is claimed is:

1. A method for optimizing cell survival in an immunoisolation device implantation in a recipient animal, said method comprising the steps of:
(a) loading cells into a first immunoisolation device;
(b) implanting said first immunoisolation device into a host animal;
(c) removing said first immunoisolation device from said host animal after a period of time;
(d) unloading the cells from said removed first immunoisolation device;
(e) expanding said unloaded cells on medium supporting growth of said cells;
(f) loading said expanded cells into a second immunoisolation device;
(g) optionally repeating steps (b)-(f) for one or more times;
wherein the loaded second immunoisolation device of step (f) contains cell lines optimized for survival in an implantation in said recipient animal.

2. The method of claim 1 wherein said implantation of said first immunoisolation device into said host at step (b) is performed in a manner consistent with the intended method of implantation with respect to said recipient.

3. The method of claim 1 wherein said host animal and said recipient animal are of different species.

4. The method of claim 1 wherein said cells are allogeneic to said recipient.

5. The method of claim 1 wherein said cells are xenogeneic to said recipient.

6. The method of claim 1 wherein said cells are syngeneic to said recipient.

7. The method of claim 1 wherein said cells are recombinantly engineered cells transformed by transfection by a vector comprising heterologous and/or homologous polynucleotide(s).

8. The method of claim 1 wherein said cells are isolated from a common clone.

9. The method of claim 1 wherein said cells secrete a polypeptide, or variant thereof, needed for the homeostasis of said recipient.

10. The method of claim 9 wherein the cells' secretion of said polypeptide, or variant thereof, is inducible by way of a secretagogue.

11. The method of claim 1 wherein said period of time in step (c) is in the range of days.

12. The method of claim 1 wherein said period of time in step (c) is in the range of weeks.

13. The method of claim 1 wherein said period of time in step (c) is in the range of months.

14. The method of claim 1 wherein steps (a)-(f) are repeated at least twice.

15. A method for selecting cells with optimal desired functionality in an immunoisolation device implantation in a recipient animal, said method comprising the steps of:
(a) loading cells having the desired functionality into a plurality of first immunoisolation devices;
(b) implanting said first immunoisolation devices into a plurality of host animals;
(c) monitoring said host animals for said cellular functionality;
(d) removing said immunoisolation devices from said host animals suggesting a predetermined level of cellular functionality;
(e) unloading the cells from said removed immunoisolation devices onto a plurality of medium supports supporting growth of said unloaded cells;
(f) expanding said unloaded cells on said medium supports;
(g) determining said medium supports that contain cells having a predetermined level of desired cellular functionality;
(h) loading said expanded cells having said predetermined level of desired cellular functionality into one or more second immunoisolation device(s); and
(i) optionally repeating steps (b)-(h) for one or more times;
wherein the loaded immunoisolation device of step (h) contains cell lines having optimal desired functionality for immunoisolation device implantation into said recipient animal.

16. The method of claim 15 wherein said implantation of said first immunoisolation devices into said host animals at step (b) is performed in a manner consistent with the intended method of implantation with respect to said recipient.

17. The method of claim 15 wherein said host animals and said recipient animal are of different species.

18. The method of claim 15 wherein said cells are allogeneic to said recipient.

19. The method of claim 15 wherein said cells are xenogeneic to said recipient.

20. The method of claim 15 wherein said cells are syngeneic to said recipient.

21. The method of claim 15 wherein said cells are recombinantly engineered cells transformed by transfection by a vector comprising heterologous and/or homologous polynucleotide(s).

22. The method of claim 21 wherein said heterologous and/or homologous polynucleotide(s) encode for a polypeptide associated with the desired functionality of the cells.

23. The method of claim 15 wherein said cells are isolated from a common clone.

24. The method of claim 15 wherein said functionality of said cell is inducible by way of a secretagogue.

25. The method of claim 15 wherein each of said first set of immunoisolation devices of step (a) are implanted into a separate host animal in step (b).

26. The method of claim 15 wherein said monitoring of said host animals at step (c) entails monitoring of blood levels of a product.

27. The method of claim 15 wherein the cells from each removed immunoisolation device in step (e) are unloaded onto a separate medium support.

28. The method of claim 15wherein steps (a)-(h) are repeated two or more times.

29. An immunoisolation system comprising:
(a) cells selected by the method of claim 15 and (b) an immunoisolation device, wherein said cells are housed within said immunoisolation device.

30. A method for optimizing cell survival in an immunoisolation device implantation in a recipient animal, said method comprising the steps of:
(a) loading cells into a first immunoisolation device;
(b) culturing said first immunoisolation device into a culture vessel;
(c) removing said first immunoisolation device from said culture vessel after a period of time;
(d) unloading the cells from said removed first immunoisolation device;
(e) expanding said unloaded cells on medium supporting growth of said cells;
(f) loading said expanded cells into a second immunoisolation device; and
(g) optionally repeating steps (b)-(f) for one or more times;
wherein the loaded second immunoisolation device of step (f) contains cell lines optimized for survival in an immunoisolation device cultured in a culture vessel.

31. The method of claim 30 wherein said cells are allogeneic to said recipient.

32. The method of claim 30 wherein said cells are xenogeneic to said recipient.

33. The method of claim 30 wherein said cells are syngeneic to said recipient.

34. The method of claim 30 wherein said cells are recombinantly engineered cells transformed by transfection by a vector comprising heterologous and/or homologous polynucleotide(s).

35. The method of claim 30 wherein said cells are isolated from a common clone.

36. The method of claim 30 wherein said cells secrete a polypeptide, or variant thereof, needed for the homeostasis of said recipient.

37. The method of claim 36 wherein the cells' secretion of said polypeptide, or variant thereof, is inducible by way of a secretagogue.

38. The method of claim 30 wherein steps (a)-(f) are repeated at least twice.

39. A method for selecting cells with optimal desired functionality in an immunoisolation device implantation in a recipient animal, said method comprising the steps of:
(a) loading cells having the desired functionality into a plurality of first vascularizing immunoisolation devices;
(b) culturing said first immunoisolation devices into a plurality of culture vessels;
(c) monitoring culture medium said culture vessels for said cellular functionality;
(d) removing said immunoisolation devices from said culture vessels suggesting a predetermined level of cellular functionality;
(e) unloading the cells from said removed immunoisolation device onto a plurality of medium supports supporting growth of said unloaded cells;
(f) expanding said unloaded cells on said medium supports;
(g) determining said medium supports that contain cells having a predetermined level of desired cellular functionality;
(h) loading said expanded cells having said predetermined level of desired cellular functionality into one or more second vascularizing immunoisolation device(s); and
(i) optionally repeating steps (b)-(h) for one or more times;
wherein the loaded immunoisolation device of step (h) contains cell lines having optimal desired functionality for immunoisolation device implantation into said recipient animal.

40. The method of claim 39 wherein said cells are allogeneic to said recipient.

41. The method of claim 39 wherein said cells are xenogeneic to said recipient.

42. The method of claim 39 wherein said cells are syngeneic to said recipient.

43. The method of claim 39 wherein said cells are recombinantly engineered cells transformed by transfection by a vector comprising heterologous and/or homologous polynucleotide(s).

44. The method of claim 39 wherein said heterologous and/or homologous polynucleotide(s) encode for a polypeptide associated with the desired functionality of the cells.

45. The method of claim 39 wherein said cells are isolated from a common clone.

46. The method of claim 39 wherein said functionality of said cell is inducible by way of a secretagogue.

47. The method of claim 39 wherein each of said set of first vascularizing immunoisolation devices of step (a) are cultured in a separate culture vessel in step (b).

48. The method of claim 39 wherein said monitoring of said host animals at step (c) entails monitoring of levels of a product.

49. The method of claim 39 wherein the cells from each removed immunoisolation device in step (e) are unloaded onto a separate medium support.

50. The method of claim 39 wherein steps (a)-(h) are repeated two or more times.

51. An immunoisolation system comprising:
(a) cells selected by the method of claim 15 or 39 and (b) an immunoisolation device, wherein said cells are housed within said immunoisolation device.
